(19) United States
(12) Patent Application Publication
(10) Pub. No.: US 2026/0199390 A1
(43) Pub. Date: Jul. 16, 2026

(54) PREBIOTIC COMPOSITION FOR STABILIZING A HEALTHY GUT MICROBIOTA

(71) Applicant: NUTRILEADS B.V., Wageningen (NL)

(72) Inventors: Annick Marie Esther MERCENIER, Wageningen (NL); Ruud ALBERS, Wageningen (NL)

(73) Assignee: NUTRILEADS B.V., Wageningen (NL)

(21) Appl. No.: 19/130,006

(22) PCT Filed: Nov. 15, 2023
(86) PCT No.: PCT/EP2023/081967

§ 371 Date: May 14, 2025

(30) Foreign Application Priority Data
Nov. 15, 2022 (EP) 22207551

(51) Int. Cl. A61K 31/732; A61K 31/716; A61K 31/717; A61K 31/718; A61P 1/00

(52) U.S. Cl. CPC A61K 31/732; A61K 31/716; A61K 31/717; A61K 31/718; A61P 1/00

(57) ABSTRACT

The inventors have discovered that a healthy gut microbiota in a subject may be stabilized by orally administering a prebiotic composition containing at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1

### TECHNICAL FIELD OF THE INVENTION

The present invention relates to the use of a prebiotic composition for stabilizing a healthy gut microbiota. Said prebiotic composition comprises rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1.

### BACKGROUND OF THE INVENTION

The human gastro-intestinal tract provides a habitat and nutrition to a large and diverse ecosystem of microbial communities that play a crucial role in digestion, metabolism and modulation of immune function, and that have a significant impact beyond the gastrointestinal tract. It is now well established that a healthy gut microbiota contributes to the overall health of the host.

Composition and stability of the microbiota are influenced by the genetic background of the host, by environmental conditions or stressors that include, for example, diet, lifestyle, use of medicaments—e.g. antibiotics—, and developmental stage (age) of the host. This translates into a permanent and complex interaction between the host and the main components of the local microbial ecosystem. These components include the microbiota, the host immune system, the local epithelial barrier and, in case of the gut, the enteric nervous system.

Consumption of probiotics and/or prebiotics can have a favorable impact on the intestinal microbiota. Probiotics are “live micro-organisms which, when administered in adequate amounts, confer a health benefit on the host” (definition of World Health Organization). Prebiotics are nondigestible food ingredients that beneficially affect the host by selectively stimulating the growth and/or activity of one or a limited number of microbial species in the gastro-intestinal tract.

Most known prebiotics are simple oligomers of identical sugars (such as fructose, galactose or arabinose) linked by glycosidic bonds. These stimulate the selective outgrowth of microbial species that have the metabolic capabilities to (rapidly) ferment these relatively simple substrates to produce beneficial metabolites such as short chain fatty acids. Typical side effects of the use of such easily fermentable substrates include intestinal discomfort, flatulence and regurgitation. These side effects are caused by the rapid fermentative production of gasses.

WO 2011/069781 describes a polysaccharide that is capable of modulating immune response, said polysaccharide being obtained from plants of the species Camellia sinensis, wherein the backbone of the polysaccharide comprises alternating rhamnogalacturonan-I domains and alpha(1,4)-linked polygalacturonic acid or alpha(1,4)-linked oligogalacturonic acid domains, wherein the molar ratio of galacturonic acid residues to rhamnosyl residues in the backbone of the polysaccharide ranges from 2.5:1 to 1:1, and wherein the polysaccharide has a molecular weight of at least 70 kDa.

WO 2012/148277 describes a preparation having a dry matter content of at least 20 wt. %, said preparation containing at least 50% by weight of dry matter of a mixture of pectic polysaccharides, including at least 20%, calculated by weight of the pectic polysaccharides, of rhamnogalacturonan-I pectins having a molecular weight of more than 40 kDa, said mixture of pectic polysaccharides being characterized by:
a degree of methylation of the galacturonic acid residues of not more than 20%;
a degree of acetylation of the galacturonic acid residues of not more 20%;
   wherein the preparation does not form a gel when it is diluted with an aqueous solution of 50 mM ammoniumbicarbonate to a solids content of 2.5 wt. %. Also described is the use of this preparation as a medicament to modulate immune response.

WO 2020/0048609 describes a prebiotic composition for use in a method of therapeutic or prophylactic treatment of disorders associated with disturbed composition or functionality of the intestinal microbiome in a subject, said use comprising oral administration of the prebiotic composition to the subject, wherein the composition contains at least 0.1% by weight of dry matter of RG-I polysaccharides originating from fruit, carrot, pea, chicory or sugar beet, said RG-I polysaccharides having a molecular weight in excess of 15 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1.

Van den Abbeele et al., A Novel Non-Digestible, Carrot-Derived Polysaccharide (cRG-I) Selectively Modulates Human Gut Microbiota while Promoting Gut Integrity: An Integrated In Vitro Approach, Nutrients 2020, 12, 1917; doi:10.3390/nu12071917, describes three different in vitro models wherein the prebiotic properties of carrot RG-I (cRG-I) were assessed.

McKay et al., Development of an affordable, sustainable and efficacious plant-based immunomodulatory food ingredient based on bell pepper or carrot RG-I pectic polysaccharides, Nutrients 2021, 13, 963, https://doi.org/10.3390/nu13030963, describes an in vitro fermentation assay wherein the prebiotic properties of cRG-I and bell pepper RG-I were assessed, by measuring produced short chain fatty acids and changes in microbial species at phylum level.

Van den Abbeele et al., Consistent Prebiotic Effects of Carrot RG-I on the Gut Microbiota of Four Human Adult Donors in the SHIME® Model despite Baseline Individual Variability, Microorganisms 2021, 9, 2142, https://doi.org/10.3390/microorganisms9102142, describes an assessment of the impact of cRG-I on the gut microbiota composition and function upon repeated administration using the M-SHIME technology (3 weeks of cRG-I dosing 3 times per day leading to a concentration of <5 g/d). Consistent effects across four simulated adult donors included enhanced levels of acetate (+21.1 mM), propionate (+17.6 mM), and to a lesser extent butyrate (+4.1 mM) and consistent microbial changes across the different donors by the end of the experiment

Cantu-Jungles et al. (New View on Dietary Fiber Selection for Predictable Shifts in Gut Microbiota, ASM Journals mBio (2020), 11(1) e02179019) propose that dietary fibers can be classified hierarchically according to their specificity toward gut microbes. Highly specific fibers harbor chemical and physical characteristics that allow them to be utilized by only a narrow group of bacteria within the gut, reducing competition for that substrate. The use of such fibers as prebiotics targeted to specific microbes would result in predictable shifts independent of the background microbial composition.

### SUMMARY OF THE INVENTION

The gut microbiota of healthy subjects is continuously adapting and responding to external stressors/stimuli and influenced strongly by diet. The inventors have unexpectedly found that the stability of the gut microbiota of healthy subjects can be improved by oral administration of RG-I polysaccharides. More particularly, the inventors have found that administration of RG-I polysaccharides helps to shape a gut microbiota that exhibits wide species diversity whilst at the same time being less sensitive to distorting factors, such as anti-microbial diet components, medication or stress, and is therefore more resilient.

The present invention thus pertains to a prebiotic composition for use in stabilizing a healthy gut microbiota in a subject by selective prebiotic stimulation of at least one taxonomic group of commensal bacteria, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1; said use comprising oral administration of 0.1-5 grams of RG-I polysaccharides per day to the subject during a period of at least 3 days.

The stabilization of a healthy gut microbiota that is achieved by the present invention entails a reduced intra-individual variability of the gut microbiota and a reduced inter-individual variability of the gut microbiota within a group of subjects.

Although the inventors do not wish to be bound by theory, it is believed that the stabilizing effect on the gut microbiota that is achieved by oral administration of RG-I polysaccharides is associated with the medium to high specificity of RG-I polysaccharide as a dietary fiber. The inventors found that specifically medium to high specificity fibers are suited for stabilizing a healthy gut microbiota. Low specificity fibers, for example inulin, are utilized by most of the microbial species in the gut microbiota and therefore maintain the microbiota diversity—as it was before being exposed to inulin—leading to large interpersonal differences between subjects. Whereas high specificity fibers, for example xanthan, are only utilized by a narrow spectrum of microbial species, which are not consistently present in each subject's microbiota, so will therefore lead to responders and non-responders as xanthan will only be fermented if the specific species able to utilize xanthan are present in the gut microbiota.

This medium to high specificity of the RG-I polysaccharide offers the advantage that the impact on the gut microbiota of orally administered RG-I polysaccharide is consistent and predictable as it increases the abundance of a subset of bacteria commonly present in healthy human adults thus resulting in a remarkable inter-individual microbiota homogenization effect, i.e. reducing inter-individual variability. This effect on the stabilization of a healthy gut microbiota is a distinctive effect next to the prebiotic properties of RG-I polysaccharide, since prebiotics such as inulin and xanthan do not have this stabilization effect.

Reduced intra-individual variability in the microbiota over time is advantageous as fluctuations in the composition of the microbiota in response to various stressors can give rise to intestinal discomfort, or variations in the metabolization and/or uptake of nutrients, nutritional supplements and/or drugs.

Reduced inter-individual variability in the microbiota offers the advantage that it becomes easier to design e.g. a nutritional supplement or drug that works well for most individuals. Considerable inter-individual variability in groups of healthy subjects could lead to variable effectiveness or incorrect recommended dosages for nutritional supplement/drugs or could lead to inconclusive results in human intervention studies since the tested compounds “land” in a non-homogenous group of subjects all characterized by their individual microbiota.

The advantageous impact on gut microbiota of orally administered RG-I polysaccharides may be enhanced by combining RG-I polysaccharides with one or more other medium to high specificity fibers or high specificity fibers. Accordingly, the invention further pertains to a prebiotic composition comprising:
at least 0.1% by weight of dry matter of rhamnogalacturonan I (RG-I) polysaccharides as defined above; and
at least 0.1% by weight of dry matter of a prebiotic fiber selected from beta-glucan, arabinoxylan, type 2 resistant starch and combinations thereof.

### DETAILED DESCRIPTION OF THE INVENTION

Accordingly, a first aspect of the invention relates to a prebiotic composition for use in stabilizing a healthy gut microbiota in a subject by selective prebiotic stimulation of at least one taxonomic group of commensal bacteria, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1, said use comprising oral administration of 0.1-5 grams of RG-I polysaccharides per day to the subject during a period of at least 3 days.

For some jurisdictions, the invention may also be worded as a method of stabilizing a healthy gut microbiota in a subject by selective prebiotic stimulation of at least one taxonomic group of commensal bacteria, said method comprising the administration of a prebiotic composition, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1, said method comprising oral administration of 0.1-5 grams of RG-I polysaccharides per day to the subject during a period of at least 3 days.

For some jurisdictions, the invention may also be worded as a use of RG-I polysaccharides in the manufacture of a prebiotic composition for stabilizing a healthy gut microbiota in a subject by selective prebiotic stimulation of at least one taxonomic group of commensal bacteria, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1, said use comprising oral administration of 0.1-5 grams of RG-I polysaccharides per day to the subject during a period of at least 3 days.

For some jurisdictions, the invention may also be worded as a use of a prebiotic composition for stabilizing a healthy gut microbiota in a subject by selective prebiotic stimulation of at least one taxonomic group of commensal bacteria, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1, said use comprising oral administration of 0.1-5 grams of RG-I polysaccharides per day to the subject during a period of at least 3 days.

In some jurisdictions administering a prebiotic composition to a subject is considered non-therapeutic. In those instances the invention may be worded as defined above by way of a method comprising administering a prebiotic composition. For clarity, the method can also be defined as a non-therapeutic method. By definition, the words “non-therapeutic” exclude any therapeutic effect.

The term “microbiota” used herein refers to the collection microorganisms that are found within a specific environment. Gut microbiota refers to the collection of microorganisms inhabiting the gut.

The term “intra-individual variability” as used herein refers to the variability of the gut microbiota within the same subject overtime.

The term “inter-individual variability” as used herein refers to the variability of the gut microbiota when comparing the gut microbiota within a group of subjects.

The term “diversity index” as used herein refers to is a quantitative measure that reflects how many different species are present in a given gut microbiota. Examples of suitable diversity indexes are Bray-Curtis dissimilarity; weighted, unweighted, or generalized UniFrac distance; Jaccard distance; Shannon diversity index; and reciprocal Simpson diversity index.

The term “low specificity fiber” as used herein refers to non-digestible fibers harboring chemical and physical characteristics that allow them to be utilized as substrate by a large variety of commensal gut bacteria, e.g. inulin.

The term “medium to high specificity fiber” as used herein refers to non-digestible fibers harboring chemical and physical characteristics that allow them to be utilized as substrate by small community of gut commensals commonly harbored by healthy subjects, e.g. RG-I polysaccharides or beta-glucans.

The term “high specificity fiber” as used herein refers to non-digestible fibers harboring chemical and physical characteristics that allow them to be utilized as substrate by only a small sub-set of the gut commensal bacteria not harbored by all healthy individuals, e.g. xanthan.

Unless indicated otherwise, the term “or” also encompasses “and”. In other words, the phrase “composition comprising A or B” encompasses compositions containing A and B.

The subject is preferably a mammalian subject, more preferably a human subject.

The use of the prebiotic composition according to the present invention preferably stimulates the growth of at least one taxonomic group of commensal bacteria selected from the phyla Bacteroidetes, Actinobacteria, Firmicutes and combinations thereof.

In a preferred embodiment, the present use reduces intra-individual variability of the gut microbiota in a subject. The change in intra-individual variability may be suitably determined by:
i. taking a faecal sample from the same subject at different time points, wherein a first faecal sample is taken before the start of oral administration of the prebiotic composition and a second faecal sample is taken at least 3 days after the start of oral administration of the prebiotic composition;
ii. determining the microbiota composition in these faecal samples; and
iii. calculating a diversity index to measure the (dis)similarity between the microbiota composition of these faecal samples.

In accordance with another preferred embodiment, the present use reduces inter-individual variability of the gut microbiota within a group of subjects. The inter-individual variability may be suitably determined by:
i. taking a faecal sample from each of the subjects in the group;
ii. determining the microbiota composition in these faecal samples; and
iii. calculating a diversity index to measure the (dis)similarity between the microbiota composition in these faecal samples.

Preferably, the change in the inter-individual variability within a group of subjects is determined by comparing the inter-individual variability of the group before the group started oral administration of the prebiotic composition to the inter-individual variability of the group at least 3 days after the group started the oral administration of the prebiotic composition.

Preferably, the diversity index is selected from Bray-Curtis dissimilarity, unweighted UniFrac distance, weighted UniFrac distance, and generalized UniFrac distance. More preferably, for determination of intra-individual variability the Bray-Curtis dissimilarity is used as diversity index. Alternatively, more preferably, for determination of inter-individual variability, the generalized UniFrac distance is used as diversity index.

Preferably, the present use comprises oral administration of a nutritional supplement or a drug before, concurrent with or after the oral administration of the prebiotic composition, said nutritional supplement or drug having an impact on the gut microbiota or requiring metabolization by the gut microbiota for its efficacy. As used herein “oral administration of a nutritional supplement or a drug before, concurrent with or after the oral administration of the prebiotic composition” refers to the nutritional supplement or drug administration taking place in a time interval of −8 hours to +8 hours with respect to the time of oral administration of the prebiotic composition (0 h).

In a preferred embodiment, the efficacy of the nutritional supplement or drug in the subject is improved by the reduced intra-individual variability of the gut microbiota. In another preferred embodiment, the reduced inter-individual variability of the gut microbiota within a group of subjects is applied in a method of assessing the efficacy of a nutritional supplement or drug in a group of subjects.

The RG-I polysaccharides of the present invention are a type of pectic polysaccharides (or pectin). Pectin is a structural heteropolysaccharide that is present in the primary cell walls of terrestrial plants.

Pectic polysaccharides are a heterogeneous group of polysaccharides comprising varying amounts of the following polysaccharide domains:
(i) homogalacturonan (HG),
(ii) xylogalacturonan (XG),
(iii) apiogalacturonan (AG)
(iv) rhamnogalacturonan-I (RG-I), and
(v) rhamnogalacturonan-II (RG-II).

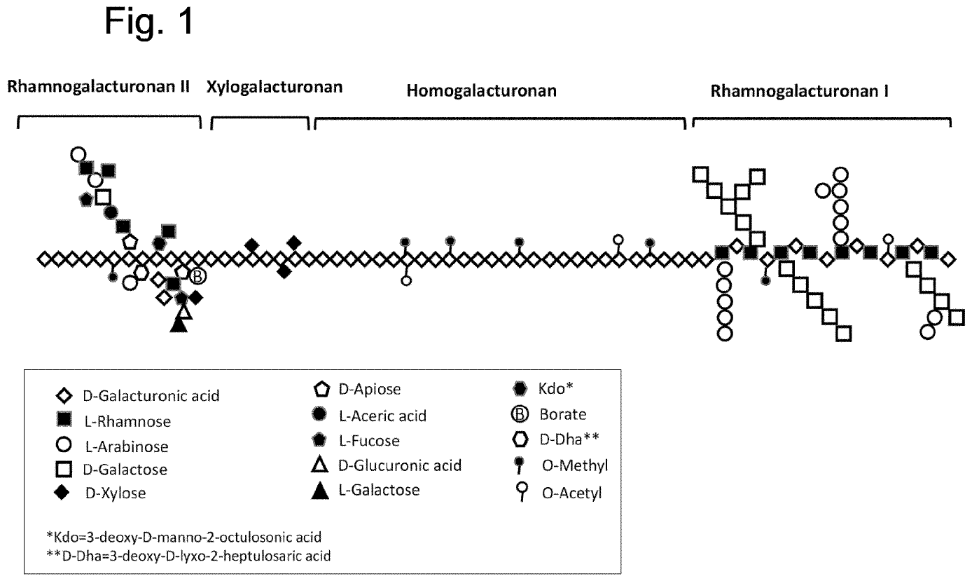

FIG. 1 provides a schematic representation of the structure of pectic polysaccharides, including the aforementioned 5 polysaccharide domains. It is noted that the polysaccharide domains AG, XG and RG-II typically represent only a very minor fraction of pectic polysaccharides.

The polysaccharide domains HG, AG, XG and RG-II each comprise a backbone that consists of a linear chain of α-(1-4)-linked D-galacturonic acid monosaccharide units (GalA).

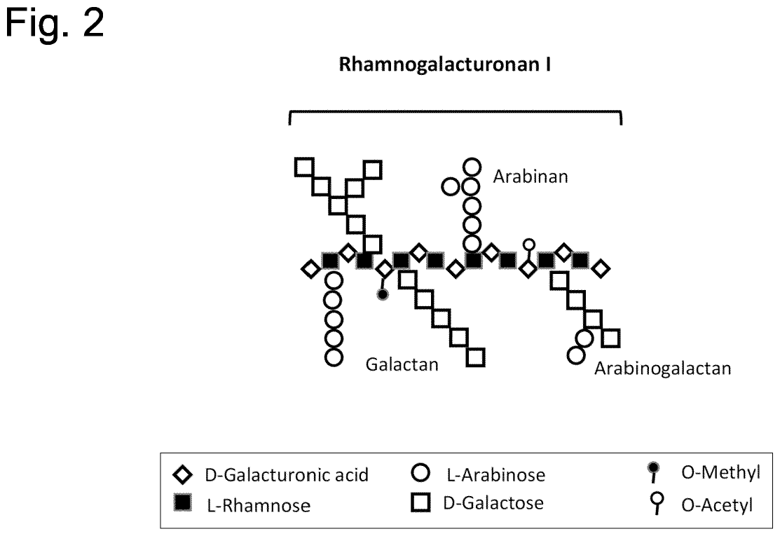

Only RG-I comprises a backbone that consists of a linear chain of the repeating disaccharide units: 4)-α-D-galacturonic acid-(1,2)-α-L-rhamnose-(1. A schematic representation of the structure of RG-I is shown in FIG. 2.

Pectic polysaccharide composition and fine structure vary depending on the plant source and the extraction conditions applied. The homogalacturonan domain can have a length of up to about 100 consecutive GalA residues. The RG-I domain containing the side chains is usually called the ‘ramified region’ or ‘hairy region’, while the homogalacturonan domain (connected to RG-I domains) is not typically substituted with glycosides or glycosidic side chains.

The GalA residues in RG-I are linked to the rhamnose (Rha) residues via the 1 and 4 positions, while the Rha residue is linked to the GalA residue via the anomeric and 2-OH positions. In general about 20-80% of the Rha residues is branched at the 4-OH position (depending on the plant source and the method of isolation), with neutral and acidic side chains. These side chains consist of Ara and Gal residues linked in various manners, constituting polymers known as arabinans, arabinogalactan I (AG-I) or arabinogalactan II (AG-II). AG I is composed of a beta-(1,4)-linked D-Gal backbone with substitutions at 3-OH of alpha-L-arabinosyl groups; the Gal backbone can have interspacing alpha(1,5)-L-Ara units. AG-II consists of highly ramified galactan with predominantly interior beta(1,3)-linked D-Gal with substitutions of short (1,6)-linked chains exteriorly. The latter has further attachments of (1,3)- and/or alpha(1,5)-linked L-Ara. The oligosaccharide side chains may be linear or branched.

The term “branched polysaccharide” as used herein refers to a polysaccharide comprising a linear backbone chain of monosaccharide units bound together by glycosidic linkages, wherein at least one of the monosaccharide units within the backbone chain carries a sidechain of one or more glycosidically linked monosaccharide units.

The terms “backbone chain” and “backbone” are synonyms.

The term “pectic polysaccharide” as used herein refers to optionally branched polysaccharides having a molecular weight in excess of 10 kDa and comprising a backbone that consists of galacturonic acid residues or a combination of galacturonic acid residues and rhamnose residues, said rhamnose residues being contained in alpha(1→4)-galacturonic-alpha(1→2)-rhamnose residues.

The term “stretch” as used herein refers to a sequence of two or more glycosidically linked monosaccharide units within the backbone of a polysaccharide, excluding any sidechains that are attached thereto.

The term “domain” as used herein refers to a stretch plus any sidechains that are attached to said stretch.

The term “rhamnogalacturonan-I stretch” or “RG-I stretch” refers to a stretch consisting of galacturonic acid (GalA) and rhamnose (Rha) pairs, wherein the GalA residues are linked to the Rha residues via the 1 and 4 positions, while the Rha residues are linked to the GalA residue via the anomeric and 2-OH positions, i.e. alternating alpha(1→4)-galacturonic-alpha(1→2)-rhamnose residues. The RG-I domain can comprise side chains such as, for example galactan, arabinan and arabinogalactan side chains.

The term “rhamnogalacturonan-I polysaccharide” or “RG-I polysaccharide” refers to optionally branched pectic polysaccharides that comprise a backbone that contains one or more rhamnogalacturonan-I stretches. The backbone of RG-I polysaccharide may optionally have one or more side chains. These sidechains consist of residues of arabinose and/or galactose.

The term “alpha(1,4)-linked galacturonic acid stretch” refers to a stretch consisting of alpha(1→4)-galacturonic residues.

The HG domains, XG domains, AG and RG-II domains that are optionally present in the RG-I polysaccharides of the present invention comprise a backbone that consists of a linear chain of two or more α-(1-4)-linked D-galacturonic acids.

HG domains do not contain any sidechains. The carboxyl groups of galacturonic acid residues within the backbone of HG domains may be esterified. Esterified galacturonic acid may occur in the form of the methyl ester or acetyl ester.

The backbone of XG domains contains one or more sidechains in the form of D-xylose.

The backbone of AG domains contains one or more sidechains that are composed of one or more D-apiose residues.

The backbone of RG-II contains one or more side chains that are not exclusively composed of D-xylose or D-apiose. The carboxyl groups of galacturonic acid residues within the backbone of RG-II domains may be esterified. Galacturonic acid may be esterified either by methyl or acetyl groups, forming methyl or acetyl esters, respectively.

The terminology “degree of acetylation” refers to the number of acetyl residues per galacturonic acid residue, expressed as a percentage.

The terminology “degree of methylation” refers to the number of methyl residues per galacturonic acid residue, expressed as a percentage.

The concentration of different polysaccharides and their monosaccharide composition can be determined by analytical techniques known to the skilled person. After acid hydrolysis (methanolysis), the monosaccharide composition of neutral sugars, can suitably be determined by High Performance Anion Exchange Chromatography combined with Pulse Amperometric Detection (HPAEC-PAD).

Uronic acids (Galacturonic acid being the dominant form of uronic acids) can be determined using the colorimetric m-hydroxydiphenyl assay.

The molecular size distribution can be determined by High Performance Size-Exclusion Chromatography (HPSEC) using refractive index (RI) detection (concentration).

The above mentioned analytical methods are described in: Analytical Biochemistry Vol. 207, Issue 1, 1992, pg 176 (for neutral sugar analysis) and in Mol. Nutr. Food Res., Vol 61, Issue 1, 2017, 1600243 (for the Uronic acid analysis and the molecular size distribution).

All percentages mentioned herein, unless otherwise stated, refer to the percentage by weight.

Oral administration within the context of the present use encompasses self-administration.

The present use comprises oral administration of 0.1-5 grams of the RG-I polysaccharides per day during a period of at least 3 days, preferably during a period of at least 6 days, more preferably during a period of at least 8 days, even more preferably during a period of at least 10 days, yet more preferably during a period of at least 20 days and most preferably during a period of at least 30 days. According to a particularly preferred embodiment, the use comprises oral administration of the RG-I polysaccharides during the aforementioned periods in a daily amount of 0.1 to 3 grams, more preferably 0.15 to 2.5 grams, most preferably of 0.2 to 2 grams.

The present use preferably comprises at least daily oral administration, most preferably once daily oral administration of the prebiotic composition containing the RG-I polysaccharides.

Preferably, the use comprises at least once daily oral administration of the prebiotic composition during at least 10 days, more preferably during at least 20 days, most preferably during at least 30 days.

The prebiotic composition preferably contains 0.2-75% by weight of dry matter, more preferably 0.3-50% by weight of dry matter, even more preferably 0.4-25%, by weight of dry matter and most preferably 0.5-10% by weight of dry matter of the RG-I polysaccharides.

The RG-I polysaccharides that are employed in the present use may be obtained from different crops. In a preferred embodiment, the RG-I polysaccharides are obtained from one or more crops selected from fruit (including tomato), carrot, olive, pea, sugar beet, red beet, chicory, okra, soy, sunflower, rapeseed and maize. More preferably, the RG-I polysaccharides are obtained from one or more crops selected from apple, pear, citrus, carrot, sugar beet and chicory. Yet more preferably, the RG-I polysaccharides are obtained from one or more crops selected from apple, pear, carrot and chicory. Most preferably, the RG-I polysaccharides are obtained from carrot, apple and/or chicory.

The RG-I polysaccharides are preferably incorporated in the prebiotic composition in the form of a pectic polysaccharide isolate that is enriched in RG-I polysaccharides. Accordingly, in a particularly preferred embodiment, the RG-I polysaccharides represent at least 10 wt. %, more preferably at least 20 wt. %, even more preferably at least 30 wt. %, yet more preferably at least 60 wt. %, and most preferably at least 80 wt. % of the pectic polysaccharides present in the prebiotic composition.

The RG-I polysaccharides that are employed in accordance with the present invention have a backbone that comprises rhamnogalacturonan-I stretches and optionally alpha(1,4)-linked homo-galacturonic acid stretches. Preferably, the molar ratio of galacturonic acid residues to rhamnose residues in the RG-I polysaccharides does not exceed 15:1, more preferably does not exceed 12:1, and even more preferably does not exceed 10:1 and yet even more preferably does not exceed 8:1 and most preferably does not exceed 5:1.

Preferably, the RG-I polysaccharides have the following monosaccharide composition having one or more of the following features:
20-60 mol. % galacturonic acid residues, wherein the individual galacturonic acids may be methylated and/or acetyl-esterified;
8-50 mol. % rhamnose residues;
0-40 mol. % arabinose residues;
0-40 mol. % galactose residues;
molar ratio of galacturonic acid residues to rhamnose residues in the range of 5:1 to 1:1;
galacturonic acid residues, rhamnose residues, arabinose residues and galactose residues together constitute at least 85 mol. % of the monosaccharide residues in the RG-I polysaccharides.

Galacturonic acid residues typically represent 21-55%, more preferably 22-50% and most preferably 23-45% of all the monosaccharide residues contained in the RG-I polysaccharides, i.e. including the monosaccharide residues that are contained in sidechains.

Rhamnose residues typically represent 9-45%, more preferably 10-40% and most preferably 11-35% of all the monosaccharide residues contained in the RG-I polysaccharides, i.e. including the monosaccharide residues that are contained in sidechains.

Arabinose residues typically represent 4-38%, more preferably 6-36% and most preferably 8-34% of all the monosaccharide residues contained in the RG-I polysaccharides.

Galactose residues typically represent 4-42%, more preferably 8-40% and most preferably 10-38% of all the monosaccharide residues contained in the RG-I polysaccharides.

The molar ratio of galacturonic acid residues to rhamnose residues in the RG-I polysaccharides preferably is within the range of 5:1 to 1:1. More preferably, the molar ratio of galacturonic acid residues to rhamnose residues in the RG-I polysaccharides ranges from 4.8:1 to 1:1, even more preferably from 4.5:1 to 1:1, yet more preferably from 4.2:1 to 1:1, most preferably from 4:1 to 1.1:1.

The combination of galacturonic acid residues, rhamnose residues, arabinose residues and galactose residues together preferably constitutes at least 88 mol. %, more preferably at least 90 mol. % and most preferably at least 92 mol. % of the monosaccharide residues in the RG-I polysaccharides.

The arabinan side chain comprises at least one or more alpha(1,5)-linked arabinose residues and is substituted to the 4-OH position of a rhamnose residues in the RG-I domain. The arabinan side chain may be linear or branched. In case the side chain is linear, the side chain consists of alpha(1,5)-linked arabinose residues. In case the arabinan side chain is a branched side chain, one or more alpha-arabinose residues are linked to the O-2 and/or O-3 of alpha(1,5)-linked arabinoses.

The galactan side chain comprises at least one or more beta(1,4)-linked galactose residues and is substituted at the O-4 position of a rhamnose residues in the RG-I domain.

The arabinogalactan side chain is substituted at the O-4 position of a rhamnose residue in the RG-I domain and can be a type I arabinogalactan (AGI) or a type II arabinogalactan (AGII). AGI is composed of a (1→4)-β-D-Galp backbone on which substitutions by monomeric Galp units at the O-6 or at the O-3 position can occur. AGI is further substituted with α-L-Araf-p residues and/or with (1→5)-α-L-Araf short side chains. AGII is composed of α(1→3)-β-D-Galp backbone decorated with (1→6)-β-D-Galp secondary chains, which are arabinosylated.

Preferably, arabinose residues and rhamnose residues are present in the RG-I polysaccharides in a molar ratio of less than 4:1, more preferably of less than 3:1, most preferably of less than 2:1.

Galactose residues and rhamnose residues are preferably present in the RG-I polysaccharides in a molar ratio of less than 4:1, more preferably of less than 3.2:1, most preferably of less than 2.5:1.

The molar ratio of the combination of arabinose residues and galactose residues to rhamnose residues in the RG-I polysaccharides preferably is less than 7:1, more preferably less than 5:1 and most preferably less than 4:1.

The combination of galacturonic acid residues and rhamnose residues preferably constitutes at least 30 mol. %, more preferably 35-90 mol. % and most preferably 40-75 mol. % of the monosaccharide residues contained in the RG-I polysaccharides.

The RG-I polysaccharides that are employed in the present use preferably have the following monosaccharide composition:
20-60 mol. % galacturonic acid residues, wherein the individual galacturonic acids can be methylated and/or acetyl-esterified;
8-50 mol. % rhamnose residues;
0-40 mol. % arabinose residues;
0-40 mol. % galactose residues;
molar ratio of galacturonic acid residues to rhamnose residues in the range of 5:1 to 1:1; and
galacturonic acid residues, rhamnose residues, arabinose residues and galactose residues together constitute at least 85 mol. % of the monosaccharide residues in the RG-I polysaccharides.

According to a particularly preferred embodiment, the RG-I polysaccharides have the following monosaccharide composition:
21-55 mol. % galacturonic acid residues, wherein the individual galacturonic acids can be methylated and/or acetyl-esterified;
9-35 mol. % rhamnose residues;
5-35 mol. % arabinose residues;
5-40 mol. % galactose residues.

The RG-I polysaccharides are preferably obtained by partial enzymatic hydrolysis of pectin. According to a particularly preferred embodiment, the RG-I polysaccharides are obtained by enzymatic hydrolysis of pectin using one or more pectinases selected from pectin lyase (EC 4.2.2.10), pectate lyase (EC 4.2.2.2), endo-polygalacturonase (EC 3.2.1.15), exopolygalacturonase (EC 3.2.1.67 and EC 3.2.1.82). Most preferably, the RG-I polysaccharides are obtained by enzymatic hydrolysis of pectin using one or more pectinases selected from pectin lyase (EC 4.2.2.10) and endo-polygalacturonase (EC 3.2.1.15).

The prebiotic composition of the present invention preferably contains traces of one or more of the aforementioned pectinases. These pectinases may be present in the product in active and/or inactive form.

According to one preferred embodiment, the RG-I polysaccharides are obtained by enzymatic hydrolysis of pectin using endopolygalacturonase and/or exopolygalacturonase in combination with pectinesterase (EC 3.1.1.11).

According to another preferred embodiment, the RG-I polysaccharides are obtained by enzymatic hydrolysis of pectin using pectin lyase and/or pectate lyase.

The RG-I polysaccharides typically have a molecular weight of at least 15 kDa. More preferably, the RG-I polysaccharides have a molecular weight between 20 kDa and 300 kDa, most preferably between 40 kDa and 300 kDa.

The RG-I polysaccharides preferably have a degree of acetylation of at least 20%, more preferably of 30-110%, even preferably of 35-90% and most preferably of 40-70%.

The RG-I polysaccharides preferably have a degree of methylation of not more than 50%, more preferably of not more than 40% and most preferably of 10-30%.

According to another preferred embodiment, the ratio of the degree of acetylation (DA) of the RG-I polysaccharides to the degree of methylation (DM) of the RG-I polysaccharides preferably is 1 or more, more preferably 2 or more, more preferably 3 or more, and most preferably 5 or more.

Whereas pectic polysaccharides normally do not contain unsaturated galacturonic acid residues, hydrolysis of pectic polysaccharides by pectin lyase and/or pectate lyase, inevitably yields polysaccharide fragments that contain a terminal unsaturated non-reducing galacturonic acid residue. Preferably, at least 10%, more preferably at least 25% and most preferably at least 50% of the terminal non-reducing galacturonic acid residues in the RG-I polysaccharides are unsaturated galacturonic acid residues. Unsaturated galacturonic acids can easily be identified, e.g. by measuring UV absorption at 235 nm.

According to a particularly preferred embodiment, the pectic polysaccharides in the present prebiotic composition (including the RG-I polysaccharides) have the following monosaccharide composition:
20-60 mol. % galacturonic acid (GalA) residues, wherein the individual galacturonic acids can be methylated and/or acetyl-esterified;
8-50 mol. % rhamnose (Rha) residues;
0-40 mol. % arabinose (Ara) residues;
0-40 mol. % galactose (Gal) residues;
   and wherein:

$$\begin{matrix}2 \times [ Rha ] + [ Ara ] + [ Gal ] \geq 50 mol . \% ; \\ [ Gal A ] - [ Rha ] < 50 mol . \% ;\end{matrix}$$

[Rha], [Ara], [Gal] and [GalA] representing the molar concentration in mol. % of rhamnose, arabinose, galactose and galacturonic acid, respectively.

More preferably, the monosaccharide composition meets the following conditions:

$$\begin{matrix}2 \times [ Rha ] + [ Ara ] + [ Gal ] \geq 60 mol . \% ; \\ [ Gal A ] - [ Rha ] < 30 mol . \% ;\end{matrix}$$

The prebiotic composition preferably is a nutritional formulation, a food product, a dietary supplement (e.g. a pill, tablet, gummy, or a powder) or a beverage.

Advantageously, the combination of RG-I polysaccharides with one or more medium to high specificity fibers or high-specificity fibers allows for specific stimulation of a subset taxonomic groups of commensal bacteria, without stimulating a large variety of taxonomic groups present in the commensal bacteria, as a low specificity fiber would do.

Accordingly to a preferred embodiment, the present use comprises oral administration of a prebiotic fiber before, concurrent with or after the oral administration of the prebiotic composition, wherein the prebiotic fiber is selected from beta-glucan, arabinoxylan, type 2 resistant starch, and combinations thereof. More preferably, the present use comprises oral administration of 0.1-20 grams of said prebiotic fiber per day, before, concurrent with or after the oral administration of the prebiotic composition, to the subject during a period of at least 3 days. Preferably during a period of at least 6 days, more preferably during a period of at least 8 days, even more preferably during a period of at least 10 days, yet more preferably during a period of at least 20 days and most preferably during a period of at least 30 days. According to a particularly preferred embodiment, the use comprises oral administration of the prebiotic fiber during the aforementioned periods in a daily amount of 0.2 to 10 grams, most preferably of 0.3 to 5 grams. As used herein “oral administration of a prebiotic fiber before, concurrent with or after the oral administration of the prebiotic composition” refers to the prebiotic fiber administration taking place in a time interval of −8 hours to +8 hours with respect to the time of oral administration of the prebiotic composition (0 h).

Preferably, the prebiotic composition comprises at least 0.1 wt. % by weight of dry matter of the prebiotic fiber selected from beta-glucan, arabinoxylan, type 2 resistant starch, and combinations thereof.

Preferably, the prebiotic composition comprises 0.2-75 wt. %, more preferably 0.3-50 wt. %, even more preferably 0.4-25 wt. % and most preferably 0.5-10 wt. % by weight of dry matter of the prebiotic fiber.

The prebiotic composition is preferably a solid dosage unit having a weight of 200-4,000 mg and containing 100-2,000 mg of the RG-I polysaccharides and 100-2,000 mg of the prebiotic fiber.

The prebiotic composition is preferably a packaged aqueous liquid having a packaged volume of 10-250 mL and containing 100-2,000 mg of the RG-I polysaccharides and 100-2,000 mg of the prebiotic fiber.

The prebiotic fiber is preferably selected from beta-glucan, arabinoxylan and combinations thereof and more preferably the prebiotic fiber is beta-glucan.

A second aspect of the invention relates to a prebiotic composition comprising:
at least 0.1% by weight of dry matter of rhamnogalacturonan I (RG-I) polysaccharides as defined in any one of the preceding claims; and
at least 0.1% by weight of dry matter of a prebiotic fiber selected from beta-glucan, arabinoxylan, type 2 resistant starch, and combinations thereof.

Preferred embodiments of the prebiotic composition are the same as described herein before in relation to the prebiotic composition for use.

The invention is further illustrated by the following non-limiting examples.

### EXAMPLES

### Example 1—Human Clinical Trial

### Study Design

A single center, randomized, double-blind, placebo-controlled dose response study was conducted that had three arms with 0, 0.3 g/day cRG-1 and 1.5 g/d cRG-I in a parallel design. Details on design, adverse events, safety monitoring, disposition of subjects and primary outcome have been described by Lutter et al. (The Dietary Intake of Carrot-Derived Rhamnogalacturonan-I Accelerates and Augments the Innate Immune and Anti-Viral Interferon Response to Rhinovirus Infection and Reduces Duration and Severity of Symptoms in Humans in a Randomized Trial, Nutrients 2021, 13, 4395, doi:10.3390/nu113124395).

Stool samples were collected from the subjects (see Table 1) before start of cRG-I supplementation and after a 8-week period of cRG-I supplementation. The subjects were healthy during this period. The stool samples were stored at −80° C. until the microbiota composition was analyzed.

TABLE 1

| Group | cRG-I dose (g/day) | Number of subjects |
|---|---|---|
| A | 1.5 | 51 |
| B | 0.3 | 49 |
| C | 0 | 46 |

### Dietary Supplement

cRG-I, a natural extract from carrot (Daucus carota subsp. sativus), was supplied by Nutrileads (Wageningen, The Netherlands). cRG-I is a water soluble non-digestible fermentable fiber, enriched in the RG-I domain of pectin. The carbohydrate content of the extract is around 73 wt. %. The extraction method and extract characteristics (composition and structure) have been described McKay et al. (Development of an Affordable, Sustainable and Efficacious Plant-Based Immunomodulatory Food Ingredient Based on Bell Pepper or Carrot RG-I Pectic Polysaccharides, Nutrients 2021, 13, 963).

The monosaccharide composition of cRG-I was (% mol/mol): 14.3 rhamnose; 34.8 arabinose; 19.6 galactose; 0.8 fucose; 4.3 glucose; 0.9 mannose; 0.7 xylose; 25.0 galacturonic acid.

Based on these numbers, the percentages (mol/mol) of monosaccharides contained in respectively RG-I and HG can be estimated as follows:

$$\begin{matrix}\% RG - I = 2 \times [ Rha ] + [ Ara ] + [ Gal ] = 2 \times 14.3 + 34.8 + 19.6 = 83 \% \\ \% HG = [ Gal A ] - [ Rha ] = 25. - 14.3 = 10.7 \%\end{matrix}$$

The percentage of monosaccharides (mol/mol) contained in the respective backbones of RG-I and HG can be estimated as follows:

$$\begin{matrix}\% RG - I = 2 \times [ Rha ] / ( [ Rha ] + [ Gal A ] ) = 100 \% \times 2 \times 14.3 / ( 14.3 + 25. ) = 72.8 \% \\ \% HG = 100 \% - \% RG - 1 = 27.2 \%\end{matrix}$$

The dietary supplements were prepared by mixing with maltodextrin and caramel color to obtain identical powders as follows: 0, 0.3 and 1.5 g cRG-I extract, 3, 2.7 and 1.5 g maltodextrin (MALDEX 170, Tereos, Belgium), and, for each dose, 0.5 g caramel color type 1 (Natural spices, Mijdrecht, the Netherlands) to obtain sachets with 3.5 g powdered supplement of identical volume and appearance for the no, low- and high-dose groups, respectively.

Participants were instructed to take the dietary supplement, supplied as a powder in a sachet, once a day with food and drink items of choice during the first meal (preferably breakfast).

### Microbial Composition Analysis in the Stool Samples

DNA extraction: DNA was extracted from the stool samples using a NucleoSpin® 96 Soil kit (Macherey-Nagel). Bead beating was done on a Vortex-Genie 2 horizontally for 5 minutes at level 9. A minimum of one negative control was included per batch of samples from the DNA extraction and throughout the laboratory process (including sequencing). A ZymoBIOMICS™ Microbial Community Standard (Zymo Research) was also included in the analysis.

PCR: PCR was done with the forward primer S-D-Bact-0341-b-S-17 and reverse primer S-D-Bact-0785-a-A-21 (Klindworth et al. 2013) with Illumina adapters attached. These are universal bacterial 16S rDNA primers which target the V3-V4 region. The following PCR program was used: 98° C. for 30 s, 25×(98θC for 10 s, 55° C. for 20 s, 72° C. for 20 s), 72° C. for 300 s. Amplification was verified by running the products on an agarose gel. Indices were added in a subsequent PCR using the Nextera Index Kit V2 (Illumina) with the following PCR program: 98° C. for 30 s, 8×(98° C. for 10 s, 55° C. for 20 s, 72° C. for 20 s), 72° C. for 300 s. Attachment of indices was verified by running the products on an agarose gel.

Normalization and sequencing: Products from the nested PCR were pooled based on band intensity, and the resulting library was cleaned with magnetic beads. The DNA concentration of pooled libraries was measured fluorometrically. Sequencing was done on an Illumina MiSeq desktop sequencer using the MiSeq Reagent Kit V3 (Illumina) for 2×300 bp paired-end sequencing.

Bioinformatics analysis: The 64-bit version of USEARCH 10.0 (Edgar 2013), mothur 1.38 (Schloss et al. 2009), and in-house scripts were used for bioinformatics analysis of the sequence data. Following tag identification and trimming, sequences were trimmed at QS 10 and merged requiring a minimum overlap of 20 bp and a merged length of 400-500 bp. Sequences with ambiguous bases, without perfect match to the primers, homopolymer length greater than 10, or more than one expected error were discarded. Primer sequences were trimmed and sequences were strictly dereplicated, discarding clusters smaller than 5. Sequences were clustered at 97% sequence identity with USEARCH's—cluster_otus command, using the most abundant strictly dereplicated reads as centroids and discarding suspected chimeras based on internal comparison. Taxonomic assignment of OTUs was done using SINTAX with a cut-off value of 0.8 (Edgar 2016) against RDP training set v16 (Cole et al. 2014). All analyses were done on rarefied data.

Diversity indices: Diversity indices were calculated based on algorithms described in the Vegan package in R (https://cran.r-project.org/web/packages/vegan/vegan.pdf) and by Oksanen J et al. (https://rdrr.io/cran/vegan/man/vegdist.html).

### Intra-Individual Variability

Per subject a diversity index was calculated comparing the microbiota composition in the subject's stool sample before the start of RG-I supplementation vs. the microbiota composition in the subject's stool sample after the 8 week period of daily RG-I supplementation.

Bray-Curtis dissimilarity was used as diversity index and was calculated using the R software package described above. Bray-Curtis dissimilarity is described in Bray, J. R., and Curtis, J. T. (1957). An ordination of the upland forest communities of Southern Wisconsin. Ecol. Monogr. 27, 325-349. doi: 10.2307/1942268. The Bray-Curtis dissimilarity is bounded between 0 and 1, where 0 means the microbiota composition is the same in both stool samples (that is they share all the species), whereas 1 means that the two stool samples do not share any species.

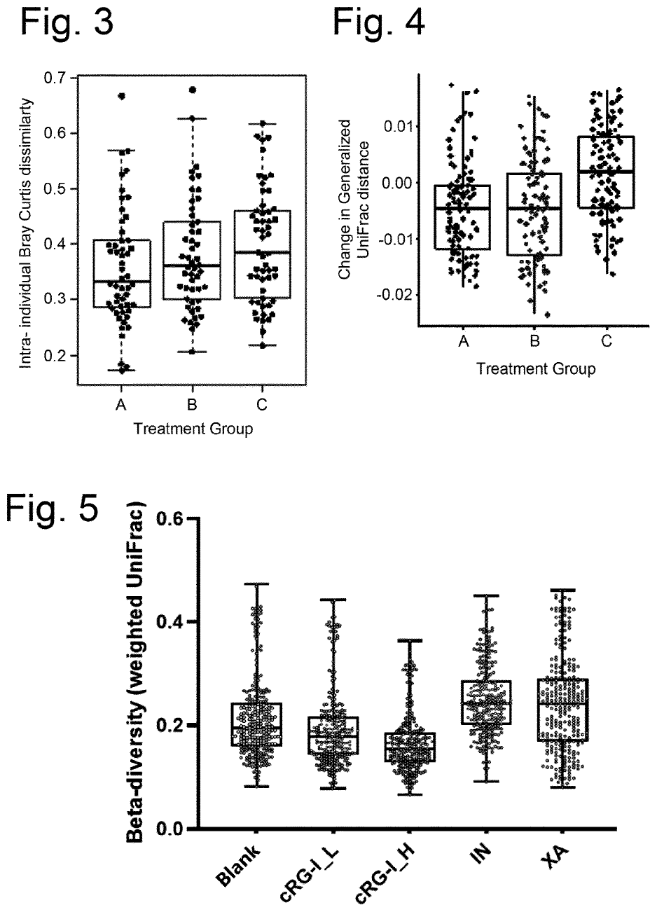

The intra-individual Bray-Curtis dissimilarity results are shown in FIG. 3 for the three treatment groups. Both group A and B, corresponding to 1.5 and 0.3 g cRG-I supplementation per day, had a lower mean intra-individual Bray-Curtis dissimilarity, compared to control group C which did not consume cRG-I.

### Inter-Individual Variability

Per subject generalized UniFrac distance was calculated by comparing the microbiota composition of a subject's stool sample to the microbiota composition in the stool samples of the other subjects within the same subject group (A, B, or C) and same timepoint (before or after supplementation). An average generalized UniFrac distance was calculated for each subject for both time points (before or after supplementation) and compared to each other. This was done for each subject.

Generalized UniFrac distance was used as diversity index and was calculated using the R software package described above. Generalized UniFrac distance is described in Chen et al., Associating microbiome composition with environmental covariates using generalized UniFrac distances, Bioinformatics, Volume 28, Issue 16, 15 Aug. 2012, Pages 2106-2113, https://doi.org/10.1093/bioinformatics/bts342. Generalized UniFrac distance was calculated with α=0.5 (a is a constant that determines how much weight is given to changes in abundance when calculating UniFrac distances). Lower values are associated with higher similarity, while higher values indicate more dissimilarity.

FIG. 4 shows that the average generalized UniFrac distance from a subject to other subjects within the same group is lower at the end of the RG-I supplementation period than before the start of cRG-I supplementation. Both group A and B, corresponding to 1.5 and 0.3 g cRG-I supplementation per day, showed a mean change of −0.004 in generalized UniFrac distance when comparing before and after cRG-I supplementation, whereas in control group C, which did not consume cRG-I, the mean difference was +0.002 when comparing the UniFrac distance before and after the control supplementation. This difference was statistically significant for group A (paired t-test, p=0.022) and group B (paired t-test, p=0.034), but not group C (paired t-test, p=0.42). The inter-individual variability thus decreased in group A and B after the supplementation with cRG-I, whereas in the control group the inter-individual variability did not change.

### Example 2—Ex Vivo Data

### Stool Samples

Fresh fecal samples were collected from healthy donors, age: 25-65 y, who had no antibiotic use in the past 3 months, no gastro-intestinal disorders (cancer, ulcers, IBD), no use of probiotic, non-smoking, alcohol consumption <3 units/d and BMI<30.

### Set-Up

An ex vivo SIFR® study was performed, simulating the colonic fermentation of test products by the gut microbiota of human adults (n=24) derived from the stool samples. Samples of the colonic fermentation was collected after 48 hours of incubation with the test products.

There were 5 study arms with different test products (Table 2):
Blank=background medium+microbiota (no product)
cRG-I_L=cRG-I tested at dose of 0.3 g/d
cRG-I_H=cRG-I tested at dose of 1.5 g/d
IN=Inulin tested at dose of 1.5 g/d
XA=Xanthan tested at dose of 1.5 g/d

TABLE 2

| | |
|---|---|
| cRG-1 | Same cRG-I as described in Example 1 |
| IN = Inulin | Purchased from Sigma Aldrich, product number I2255 |
| XA = Xanthan | Purchased from Carl Roth, item number 3557 |

### Ex-Vivo SIFR®: Technology

Individual bioreactors were processed in parallel in a proprietary bioreactor management device (Cryptobiotix, Ghent, Belgium). Each bioreactor contained 5 mL of nutritional medium-fecal inoculum blend supplemented with 0.3 or 1.5 g prebiotic/L, then sealed individually, before being rendered anaerobic. Blend M0003 was used for preparation of the nutritional medium (Cryptobiotix, Ghent, Belgium). M0003 is a growth medium (pH 6.5) developed by Cryptobiotix. This medium contains amongst others, sources of carbon, nitrogen, phosphorus, sulfur, vitamins and minerals in sufficient amounts to support bacterial growth. After preparation, bioreactors were incubated under continuous agitation (140 rpm) at 37° C. for 48 h in the MaxQ™ 6000 Incubated/Refrigerated Stackable Shakers (Thermo Scientific, Thermo Fisher Scientific, Merelbeke, Belgium). Upon gas pressure measurement in the headspace, liquid samples were collected for subsequent analysis.

### Microbial Composition Analysis

Upon DNA extraction, library preparation and sequencing were performed on an Illumina MiSeq platform with v3 chemistry. The 16S rRNA gene V3-V4 hypervariable regions were amplified using primers 341F (50-CCT ACG GGN GGC WGC AG-30) and 785Rmod (50-GAC TAC HVG GGT ATC TAA KCC-30). Results were analyzed at different taxonomic levels (phylum, family and OTU level). For taxonomic analysis, the proportional data derived from sequencing (%) were corrected for the total amount of cells present in each sample (detected via flow cytometry), allowing to obtain more representative insights in the impact of interventions on the gut microbiota.

### Inter-Individual Variability

The weighted UniFrac distance was calculated within each set of 24 samples derived from the 24 donors, i.e. blank and cRG-I/IN/XA treatments at 48 h. Given that a sample of a given donor was always compared to the 23 other donors, a total of 276 comparisons were performed within each experimental group.

The weighted UniFrac distance was calculated using software package QIIME v1.9.0. Weighted UniFrac distance is as described in Lozupone et al., Quantitative and Qualitative 13 Diversity Measures Lead to Different Insights into Factors That Structure Microbial Communities, Appl Environ Microbiol. 2007 March; 73(5): 1576-1585, doi: 10.1128/AEM.01996-06. Identical communities result in a weighted UniFrac distance of 0, totally different communities result in a value of 1.

The inter-individual weighted UniFrac distance results are shown in FIG. 5 for the different experimental groups. Compared to the Blank group, the 2 dosages of cRG-I significantly lowered the weighted UniFrac distances in a dose-response fashion. While, compared to the Blank group, both IN and XA significantly increased the weighted UniFrac distances. When comparing the IN and XA group to the cRG-I group at equal dosage (1.5 g/d), the cRG-I group was significant different from both the IN and XA group. The inter-individual variability was thus reduced in the presence of cRG-I, whereas the inter-individual variability was increased in the presence of inulin or xanthan.

What is claimed is:

1. A non-therapeutic method for stabilizing healthy gut microbiota in a subject by selective prebiotic stimulation of at least one taxonomic group of commensal bacteria, said method comprising the administration a prebiotic composition, wherein the prebiotic composition contains at least 0.1% by weight of dry matter of rhamnogalacturonan I (RG-I) polysaccharides having a molecular weight in excess of 10 kDa and having a backbone consisting of galacturonic acid residues and rhamnose residues, said rhamnose residues being contained in alpha(1→4)-galacturonic-alpha(1→2)-rhamnose residues, wherein the molar ratio of galacturonic acid residues to rhamnose residues in the RG-I polysaccharides is within the range of 20:1 to 1:1;
said method comprising oral administration of 0.1-5 grams of RG-I polysaccharides per day to the subject during a period of at least 3 days,
wherein the stabilizing of healthy microbiota is by
reducing intra-individual variability of the gut microbiota in a subject over time; and/or
reducing inter-individual variability of the gut microbiota within a group of subjects.

2. The method of claim 1, wherein the method comprises oral administration of 0.2-2 grams of RG-I polysaccharides per day to the subject during a period of at least 3 days.

3. The method of claim 1, wherein the method comprises oral administration of a prebiotic fiber before, concurrent with or after the oral administration of the prebiotic composition, wherein the prebiotic fiber is selected from beta-glucan, arabinoxylan, type 2 resistant starch, and combinations thereof.

4. The method of claim 1, wherein the prebiotic composition comprises at least 0.1 wt. % by weight of dry matter of a prebiotic fiber selected from beta-glucan, arabinoxylan, type 2 resistant starch and combinations thereof.

5. The method of claim 1, wherein the RG-I polysaccharides represent at least 20 wt. % of pectic polysaccharides present in the prebiotic composition.

6. The method of claim 1, wherein the molar ratio of galacturonic acid residues to rhamnose residues in the RG-I polysaccharides does not exceed 15:1.

7. The method of claim 1, wherein the RG-I polysaccharides have the following monosaccharide composition:
20-60 mol. % galacturonic acid residues, wherein the individual galacturonic acids can be methylated and/or acetyl-esterified;
8-50 mol. % rhamnose residues;
0-40 mol. % arabinose residues;
0-40 mol. % galactose residues;
molar ratio of galacturonic acid residues to rhamnose residues in the range of 5:1 to 1:1;
galacturonic acid residues, rhamnose residues, arabinose residues and galactose residues together constitute at least 85 mol. % of the monosaccharide residues in the RG-I polysaccharides.

8. The method claim 1, wherein the RG-I polysaccharides have the following monosaccharide composition:
20-60 mol. % galacturonic acid (GalA) residues, wherein the individual galacturonic acids can be methylated and/or acetyl-esterified;
8-50 mol. % rhamnose (Rha) residues;
0-40 mol. % arabinose (Ara) residues;
0-40 mol. % galactose (Gal) residues;
and wherein:
$$\begin{matrix}2 \times [ Rha ] + [ Ara ] + [ Gal ] \geq 50 mol . \% ; \\ [ Gal A ] - [ Rha ] < 50 mol . \% ;\end{matrix}$$
[Rha], [Ara], [Gal] and [GalA] representing the molar concentration in mol. % of rhamnose, arabinose, galactose and galacturonic acid, respectively.

9. The method f claim 1, wherein the RG-I polysaccharides have a degree of acetylation of at least 20% and a degree of methylation of not more than 50%.

10. The method of claim 1, wherein the RG-I polysaccharides originate from carrot, apple or chicory.

11. A prebiotic composition comprising:
at least 0.1% by weight of dry matter of rhamnogalacturonan I (RG-I) polysaccharides as defined in claim 1; and
at least 0.1% by weight of dry matter of a prebiotic fiber selected from beta-glucan, arabinoxylan, type 2 resistant starch and combinations thereof.

12. The prebiotic composition of claim 11, wherein the prebiotic composition is a nutritional formulation, a food product, a dietary supplement or a beverage.

13. The prebiotic composition of claim 11, wherein the prebiotic fiber is beta-glucan and/or arabinoxylan.

14. The prebiotic composition of claim 13, wherein the prebiotic fiber is beta-glucan.


SEQUENCE LISTING (2 sequences)

SEQ ID NO: 1 - DNA, 17 bp, synthetic construct
  source 1..17: mol_type = other DNA
cctacgggnggcwgcag

SEQ ID NO: 2 - DNA, 21 bp, synthetic construct
  source 1..21: mol_type = other DNA
gactachvgggtatctaakcc